(19) United States
(12) Patent Application Publication
(10) Pub. No.: US 2007/0148640 A1
(43) Pub. Date: Jun. 28, 2007

(54) Methods and kits for decreasing interferences in plasma or serum containing assay samples of specific binding assays

(76) Inventors: Richard L. Scopp, Kenosha, WI (US); David M. Finley, Spring Grove, IL (US); Kevin L. Trimpe, Gurnee, IL (US); Agnieszka Lach, Chicago, IL (US); Cynthia D. Pestel, Lombard, IL (US); John M. Ramp, Gurnee, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/709,374

(22) Filed: Feb. 22, 2007

Related U.S. Application Data
(63) Continuation of application No. 09/669,082, filed on Sep. 25, 2000, now abandoned.

(51) Int. Cl. C12Q 1/70; G01N 33/53

(52) U.S. Cl. 435/5; 435/7.1

(57) ABSTRACT

Methods and kits are provided for decreasing interferences and inaccuracies due to nonoptimal sample handling of blood samples in plasma or serum containing assay samples of specific binding assays by addition of a large polycation to the assay sample during the specific binding assay.

### FIELD OF THE INVENTION

The present invention relates to an improved method for performing specific binding assays with plasma or serum samples wherein a relatively large polycation is added to the assay sample during the assay. The present invention also relates to improved specific binding assay kits for plasma or serum samples which comprise as one component of the kit a solution containing a large polycation.

### BACKGROUND OF THE INVENTION

Polycations are organic or inorganic, synthetic or naturally occurring, compounds having at least two positive charges. Examples of relatively large polycations include, but are not limited to, polylysine, polyethyleneimine and polypropyleneimine and their lower alkyl ammonium salts such as polybrene, and MERQUAT.

Polycations such as polylysine, polyarginine and polyhistidine are commercially available for use as enzyme inhibitors, as substrates in the isolation of plasma membranes, in chromosomal preparations, in microencapsulation, in sustained release delivery devices, and as drug delivery devices. Poly-L-lysine is also used as a carrier protein in the synthesis of immunogens, while poly-D-lysine is used as a carrier protein in immobilized antigen enzyme linked immunosorbent assays (ELISAs). Polycations such as poly(N-ethyl-4-vinylpyridinium have also been used, in conjunction with polyanions such as poly(methacrylate), as carriers for reactants in both ELISAs (Yazynina et al. Analytical Chemistry 1999 71(16):3538-43) and visual enzyme immunoassays (Dzantiev et al. Immunology Letters 1994 41(2-3):205-11).

Polyionic reagents including polycations have been disclosed for use in initiating non-specific binding of a substance to magnetic particles. For example, U.S. Pat. Nos. 4,935,147, 5,076,950, 5,279,936 and 5,770,388 disclose a list of exemplary polycationic reagents including polyalkylene amines such as polyethyleneimine and polypropyleneimine and their lower alkyl ammonium salts such as polybrene (N(CH₃)₂CH₂CH₂N(CH₃)₂CH₂CH₂CH₂CH₂—)ₙ, metal ions such as calcium and barium ions, aminodextrans, protamine, positively charged liposomes, polylysine, and the like for use as a chemical means for forming non-specific bonds between the substance and magnetic particles.

Polycations have also been taught to be useful in separation techniques for immunoassay of whole blood samples. WO 9936781 discloses a chromatography assay device which separates red blood cells in a sample from serum or plasma prior to movement of the serum or plasma down the chromatography column. The red blood cell separating agent used in this device is preferably a polycation comprising poly-L-lysine hydrobromide, poly-L-arginine hydrochloride, poly-L-histidine, poly(lysine, alanine) 3:1 hydrobromide, poly(lysine, arginine) 2:1 hydrobromide, poly(lysine, alanine) 1:1 hydrobromide, poly(lysine, tryptophan) 1:4 hydrobromide or particularly poly(diallyldimethylammonium chloride). However, addition of a separating agent such as a polycation directly to the assay system is taught to interfere with the system, often by aggregating other reagents and binding members in addition to the red blood cells.

Accordingly, an object of the present invention is to provide a method for decreasing interferences which result in inaccurate readings in plasma or serum containing assay samples of specific binding assays. The method comprises adding a large polycation to the plasma or serum containing assay sample during the specific binding assay.

Another object of the present invention is to provide improved specific binding assay kits for plasma and serum containing assay samples which comprise as one component of the kit a solution containing a large, polycation.

### SUMMARY OF THE INVENTION

The present invention provides a method for decreasing interferences which result in inaccurate readings in serum or plasma containing assay samples of specific binding assays comprising adding an effective amount of a large polycation to serum or plasma containing assay samples during the specific binding assay. In a preferred embodiment, the large polycation has a molecular weight of 3,000 daltons or greater. In another preferred embodiment, the large polycation is a polylysine, polyornithine, polybrene or MERQUAT.

In a more preferred embodiment, the large polycation comprises a polylysine with a molecular weight ranging between 5,200 and 11,200 daltons. In another more preferred embodiment, the large polycation comprises polylysine with a molecular weight of 8,800 daltons. In another preferred embodiment, the specific binding assay is performed on a solid phase, such as paramagnetic microparticles. In other embodiments, the specific binding assay measures thyroid stimulating hormone, free prostate specific antigen (PSA), alpha fetal protein, hepatitis B core antibody, hepatitis B surface antibody or human immunodeficiency virus.

The invention also provides a method for decreasing interferences which result in inaccurate readings in serum or plasma containing assay samples of a thyroid stimulating hormone specific binding assay comprising adding a large polycation to serum or plasma containing assay samples during the thyroid stimulatinghormone specific binding assay. In a preferred embodiment, the large polycation has a molecular weight of 3,000 daltons or greater. In another preferred embodiment, the large polycation is a polylysine, polyornithine, polybrene or MERQUAT. In a more preferred embodiment, the large polycation comprises a polylysine with a molecular weight ranging between 5,200 and 11,200 daltons. In another more preferred embodiment, the large polycation comprises polylysine with a molecular weight of 8,800 daltons. In another preferred embodiment, the specific binding assay is performed on a solid phase, such as paramagnetic microparticles.

In a most preferred embodiment, the thyroid stimulating hormone specific binding assay comprises:

a) forming a first complex by incubating a serum or plasma sample with paramagnetic microparticles coated with anti-β TSH antibody and an assay diluent which comprises a large polycation, for a time and under conditions which allow the thyroid stimulating hormone present in the sample to bind to the anti-β TSH antibody coated microparticles;

(b) forming a second complex by incubating the first complex with an acridinium labeled conjugate comprising an anti-α TSH antibody, for a time and under conditions which allow the conjugate to bind to the first complex;

(c) creating a chemiluminescent reaction in the second complex; and

(d) measuring the chemiluminescent reaction as relative light units wherein the amount of thyroid stimulating hormone in the plasma or serum sample is directly related to the measured relative light units.

The present invention also provides a method for decreasing interferences which result in inaccurate readings in serum or plasma containing assay samples of a free or total prostate specific antigen specific binding assay comprising adding a large polycation to serum or plasma containing assay samples during the free or total prostate specific antigen specific binding assay. In a preferred embodiment, the large polycation is a polylysine or polyornithine. In another preferred embodiment, the free prostate specific antigen (PSA) specific binding assay comprises:

(a) forming a first complex by incubating a serum or plasma sample with paramagnetic microparticles coated with an antibody specific for free PSA, for a time and under conditions which allow the free PSA present in the sample to bind to the antibody coated microparticles;

(b) forming a second complex by incubating the first complex with an acridinium labeled conjugate comprising an anti-PSA antibody, for a time and under conditions which allow the conjugate to bind to the first complex;

(c) creating a chemiluminescent reaction in the second complex; and

(d) measuring the chemiluminescent reaction as relative light units wherein the amount of prostate specific antigen in the plasma or serum sample is directly related to the measured relative light units.

In another preferred embodiment the total PSA specific binding assay comprises:

(a) forming a first complex by incubating a serum or plasma sample with paramagnetic microparticles coated with an antibody which binds both free and complexed PSA, for a time and under conditions which allow the PSA present in the sample to bind to the antibody coated microparticles;

(b) forming a second complex by incubating the first complex with an acridinium labeled conjugate comprising an anti-PSA antibody, for a time and under conditions which allow the conjugate to bind to the first complex;

(c) creating a chemiluminescent reaction in the second complex; and

(d) measuring the chemiluminescent reaction as relative light units wherein the amount of prostate specific antigen in the plasma or serum sample is directly related to the measured relative light units.

The present invention also provides an improved specific binding assay kit for plasma and serum samples comprising a solution containing a large polycation. In a preferred embodiment, the large polycation has a molecular weight of 3,000 daltons or greater. In another preferred embodiment, the large polycation is a polylysine, polyornithine, polybrene or MERQUAT. In a more preferred embodiment, the improved specific binding assay kit comprises a specific binding assay which measures thyroid stimulating hormone, free prostate specific antigen, alpha fetal protein, Hepatitis B core antibody, Hepatitis B surface antibody or human immunodeficiency virus.

The present invention also provides an improved kit for detection of thyroid stimulating hormone comprising:

(a) mouse, monoclonal anti-β TSH coated microparticles;

(b) mouse, monoclonal anti-α TSH acridinium-labeled conjugate; and

(c) a modified TSH assay diluent comprising a large polycation. Preferably, the large polycation is a polylysine having a molecular weight from 5,200 to 11,200 daltons.

The present invention also provides an improved kit for detection of free prostate specific antigen comprising:

(a) microparticles comprising a monoclonal antibody specific to free PSA in a diluent comprising a large polycation;

(b) mouse, monoclonal anti-PSA acridinium-labeled conjugate. Preferably, the large polycation is a polylysine or polyornithine.

### DETAILED DESCRIPTION OF THE INVENTION

Non-optimal serum or plasma sample preparation techniques including, but not limited to, inadequate centrifugation, incomplete clotting time, and exposure to thermal stress, have been found to cause interferences in plasma or serum containing assay samples which lead to inaccurate readings in specific binding assays. It has now been found that addition of a large polycation to a plasma or serum containing assay sample during the specific binding assay decreases or eliminates these interferences so that accurate readings can be obtained.

For purposes of the present invention, by “large” polycation it is meant a polycation with a molecular weight of approximately 3,000 daltons or greater. Examples of large polycations useful in the present invention include, but are not limited to, polylysines with a molecular weight ranging between 5,200 and 11,200, polyornithine with a molecular weight of 5300, polybrene with a molecular weight ranging between approximately 4,000 and 6,000 daltons, and MERQUAT with a molecular weight of approximately 4,000,000 daltons. The polycation can be added during the immunoassay as a separate reagent. Alternatively, the polycation can be incorporated into an assay specific diluent.

The amount of polycation used in an assay may vary depending on the type and its molecular weight. Generally, however, the amount used is a quantity which is effective at achieving the desired result, i.e. eliminating interference, without detrimentally affecting other assay parameters (such as sensitivity, specificity, etc.). By way of example, polycations such as polylysines, polyornithines, polyarginines, and polyhistidines at final concentrations ranging from about 0.005% to about 1% weight/volume (wt/vol) may be used. More preferably, polylysines ranging from about 0.01% to about 0.5% wt/vol are used. Even more preferably, polylysines ranging from about 0.1% to about 0.5% wt/vol are used. For a polylysine with a molecular weight of 8,800 daltons, a concentration of about 0.25% is preferred. For polybrene, concentrations ranging from about 0.2% to 1% wt/vol are preferred. For MERQUAT, concentrations ranging from about 0.15% to about 0.30% are preferred.

While higher concentrations of a polycation may still be effective at decreasing interferences in the sample, it is believed that the higher viscosity resulting from addition of some polycations may cause carryover, particularly in high throughput automated specific binding assay systems. However, those of ordinary skill in the art could easily determine the proper concentration suitable for a particular assay.

The polycations of the present invention may be used in any type of specific binding assay that tests for the presence of an analyte (such as an antigen or antibody) in a serum or plasma sample, including but not limited to sandwich and competitive type immunoassays. Such immunoassays may utilize reagents comprising a polyclonal or monoclonal antibody, fragments of said antibodies (such as an Fab′2 fragment) or combinations of polyclonal, monoclonal and antibody fragments. Typically in such assays, a labeled reagent (such as a labeled antigen or antibody) is used for detecting and/or quantitating an analyte of interest. Such labels include, without limitation, enzymatic, fluorescent, chemiluminescent, and radioactive labels. The manner of making and using all types of immunoassays as well as the reagents and/or labeled reagents used in such assays are well know to routine practitioners in the art.

One embodiment of the present invention relates to an improved specific binding assay for measuring TSH in serum or plasma samples. In a preferred embodiment, the TSH specific binding assay comprises a modified ARCHITECT TSH assay format (Abbott Laboratories, Abbott Park, Ill. 60035-6050) wherein a large polycation with a molecular weight of approximately 3,000 daltons or greater is added to the assay sample during the assay, i.e. before or during the incubation of the sample with the solid phase. In this embodiment, it is preferred that the polycation be a polylysine with a molecular weight ranging between 5,200 and 11,200 daltons, with a polylysine having a molecular weight of approximately 8,800 daltons being preferred. It is also preferred that the polycation be incorporated within the TSH assay diluent which is combined with the plasma or serum sample and the TSH antibody. Preferred concentration ranges of polylysine in the TSH assay range from about 0.1% to about 1% wt/vol with 0.25% wt/vol being most preferred.

Another embodiment of the present invention relates to improved kits for performing this modified ARCHITECT TSH assay. Kits of the present invention comprise at least anti-β TSH (mouse, monoclonal) coated microparticles in a buffer, preferably TRIS buffer, and even more preferably with protein (bovine) stabilizers and antimicrobial agents as a preservative, an acridinium-labeled conjugate comprising a mouse anti-α TSH monoclonal antibody, preferably in MES (2-[N-Morpholino]ethanesulfonic acid) buffer with protein (bovine) stabilizers and antimicrobial agents as a preservative; and a modified TSH assay diluent comprising a buffer, preferably TRIS, containing a polycation, preferably a polylysine ranging in molecular weight from 5,200 to 11,200 daltons at a concentration ranging from about 0.1% wt/vol to about 0.5% wt/vol. It is preferred that this diluent comprise antimicrobial agents as preservatives. Alternatively, the polycation can be provided as a separate kit component for addition to the assay samples along with the TSH assay diluent. Kits of this embodiment of the present invention may also comprise a Multi-Assay Manual Diluent containing phosphate buffered saline solution with an antimicrobial agent as a preservative; a Pre-Trigger Solution containing 1.32% (w/v) hydrogen peroxide; a Trigger Solution containing 0.35 N sodium hydroxide; and a wash buffer containing phosphate buffered saline solution and an antimicrobial agent preservative.

A second preferred embodiment of the present invention relates to an improved specific binding assay for measuring free or total prostate specific antigen (PSA) in serum or plasma samples. In a most preferred embodiment, the PSA specific binding assay comprises a modified ARCHITECT total or free PSA assay format (Abbott Laboratories, Abbott Park, Ill. 60035-6050) wherein a large polycation with a molecular weight of approximately 3,000 daltons or greater is added to the assay with the assay sample, i.e. before or during the incubation of the sample with the solid phase.

In this embodiment, it is preferred that the polycation be a polylysine with a molecular weight ranging between 5,200 and 11,200 daltons. It is also preferred that the polycation be incorporated in the diluent of the anti-PSA coated microparticles (hereinafter “microparticle diluent”) which is combined with the plasma or serum sample. Preferred concentration ranges of polylysine in the total PSA assay range from about 0.005% to about 1% wt/vol with 0.005% wt/vol being most preferred. Preferred concentration ranges of polylysine in the free PSA assay range from about 0.01% to about 1% wt/vol with 0.01% wt/vol being most preferred.

Another embodiment of the present invention relates to improved kits for performing a modified ARCHITECT total or free prostate specific antigen (PSA) assay. A kit of the present invention comprises microparticles, coated with an anti-PSA monoclonal antibody (one that is specific for free PSA in the case of the free PSA assay and one that binds both free and complexed PSA for the total PSA assay) in a diluent which also contains a polycation. The kit also includes an acridinium-labeled conjugate comprising an anti-PSA monoclonal antibody. Preferably, the polycation is a polylysine ranging in molecular weight from about 5,200 to about 11,200 daltons at a concentration ranging from about 0.005% wt/vol-0.5% wt/vol. The buffer of the microparticle diluent preferably is a TRIS buffer and even more preferably contains protein (bovine) stabilizers and antimicrobial agents as a preservative. The acridinium-labeled conjugate is preferably in MES (2-[N-Morpholino]ethanesulfonic acid) buffer with protein (bovine) stabilizers and antimicrobial agents as a preservative. Alternatively, the polycation can be provided as a separate kit component for addition to the assay samples along with the PSA microparticle diluent. Kits of this embodiment of the present invention may also comprise a Pre-Trigger Solution containing 1.32% (w/v) hydrogen peroxide, a Trigger Solution containing 0.35 N sodium hydroxide, and a wash buffer containing phosphate buffered saline solution and an antimicrobial agent preservative.

Re-centrifugation of nonoptimally handled plasma and serum samples has also been demonstrated to be effective in decreasing interferences and restoring sensitivity and accuracy in sample measurement in specific binding assays for alpha fetal protein (AFP), Hepatitis B core antibody (HBcAb), Hepatitis B surface antibody (HBsAb), and human immunodeficiency virus (HIV). Accordingly, it is believed that addition of a large polycation to plasma or serum containing assay samples during performance of specific binding assays for these analytes will also be useful in decreasing interferences due to nonoptimal sample preparation.

The following nonlimiting examples are provided to further illustrate the present invention.

### EXAMPLES

### Example 1

### Preparation of Contaminated Plasma or Serum Samples

Blood was drawn from one volunteer into four serum separator tubes, also referred to as SST Vacutainer tubes (Becton Dickinson, Number 366510) and six ethylenediaminetetracetic acid (EDTA) Vacutainer tubes (Becton Dickinson, Number 366457). The blood was allowed to clot for 30 minutes and then spun in a centrifuge at 3,500 RPM for 10 minutes. Serum was recovered from the four SST tubes. Plasma was recovered from the six EDTA tubes. A portion of the plasma was then contaminated by addition of 60 microliters of buffy coat (including red blood cells) from the EDTA tubes.

### Example 2

### Effect of Polycations in the ARCHITECT TSH Assay

a. General Procedure: The ARCHITECT TSH assay (Abbott Laboratories, Abbott Park, Ill. 60035-6050) is a two-step immunoassay which determines the presence of thyroid stimulating hormone (TSH) in human serum and plasma using Chemiluminescent Microparticle Immunoassay (CMIA) technology with flexible assay protocols, referred to as CHEMIFLEX. In the first step, a serum or plasma sample, anti-β TSH antibody coated paramagnetic microparticles, and TSH Assay Diluent are combined. TSH present in the sample binds to the anti-TSH antibody coated microparticles. After washing, anti-α TSH acridinium labeled conjugate is added as the second step. Two solutions referred to as a Pre-Trigger and Trigger Solution, which comprise hydrogen peroxide and sodium hydroxide, respectively, are then added to the reaction mixture and the resulting chemiluminescent reaction is measured as relative light units (RLUs). A direct relationship exists between the amount of TSH in the plasma or serum sample and RLUs detected by the ARCHITECT/optical system.

b. Experimental Design: Experiments were designed in which serum and plasma samples were contaminated intentionally with red blood cells to interfere with the sensitivity of the assay (see Example 1). In separate experiments, a polycation, i.e. polylysine, polybrene or MERQUAT, then was added to the TSH Assay Diluent and combined with the serum or plasma sample (150 μL) and anti-β TSH antibody coated paramagnetic microparticles (50 μL at 0.1 % solids) in the first step of the TSH assay. The assay then was completed as described in the general procedure above.

c. Results: As Table 1 shows polylysines having an average molecular weight of 5,200, 8,800 and 11,200 were found to be effective at eliminating interferences in contaminated samples at a concentration of 0.25%.

TABLE 1

| | | TSH (uIU/mL) | TSH (uIU/mL) | |
|---|---|---|---|---|
| Type of | | of centrifuged | of uncentrifuged | % Differ- |
| Polylysine | | sample | sample | ence |
| No Polylysine | | 1.6654 | 0.1679 | 90 |
| 5200 | MW | 1.9545 | 1.9441 | 1 |
| 8800 | MW | 1.9665 | 1.9564 | 1 |
| 11,200 | MW | 1.9939 | 1.9132 | 4 |

Various concentrations of polybrene with a molecular weight of 4,000 to 6,000 daltons also were examined. Concentrations ranging from 0.2% to 1% wt/vol of polybrene were found to be effective at restoring assay sensitivity to contaminated plasma or serum samples without interfering with or altering the functional sensitivity of the TSH assay.

The polycation MERQUAT-100 having a molecular weight of about 4,000,000 daltons also restored assay sensitivity to contaminated samples without interfering with overall function of the assay at concentrations of either 0.15% or 0.30% in the TSH Assay Diluent.

### Example 3

### Effect of Polycations in the ARCHITECT free PSA Assay

Addition of a polycation to an assay sample also was demonstrated to be effective in decreasing interferences resulting from nonoptimal plasma or serum sample handling in an ARCHITECT free prostate specific antigen (PSA) assay.

a. General Procedure: The ARCHITECT Free PSA assay is a two step immunoassay to determine the presence of free PSA in human serum, using Chemiluminescent Microparticle immunoassay (CMIA) technology. In the first step, a test sample and paramagnetic microparticles, coated with a monoclonal antibody specific to free PSA, are combined. Free PSA present in the sample binds to the anti-free PSA coated microparticles. After washing, anti-PSA acridinium-labeled conjugate is added in the second step. Pre-Trigger and Trigger Solutions are then added to the reaction mixture; the resulting chemiluminescent reaction is measured as RLUs. A direct relationship exists between the amount of free PSA in the sample and the RLUs detected by the ARCHITECT/optical system. Like the TSH assay, nonoptimal preparation of the serum sample leads to interferences in measurement of fluorescence and ultimately an inaccurate reading of the levels of free PSA in the sample.

b. Experimental Design: In these experiments, a polycation, in particular, a poly-amino acid, was substituted in place of dextran sulfate in the microparticle diluent. The assay then was performed as described in the general procedure above.

c. Results: As shown in Table 2, both polylysine (ranging from 5,200 to 11,200 daltons) and polyornithine (5,300 daltons) at concentrations of 0.025% were effective at decreasing interferences in free PSA measurements caused by poor sample preparation without interfering with or altering the high functional sensitivity of the free PSA assay.

TABLE 2
Free PSA Concentration (ng/mL)

| | Dextran | poly-L- | poly-L- | poly-L- | poly-L- |
|---|---|---|---|---|---|
| Sample No. | Sulfate | lysine | ornithine | arginine | histidine |
| 30 (Spun)* | 0.699 | 0.703 | 0.697 | 0.528 | 0.558 |
| 30 (Unspun) | 0.000 | 0.666 | 0.632 | 0.528 | 0.511 |
| % Interfer- | 100% | 5% | 9% | 0% | 8% |
| ence** | | | | | |
| 31 (Spun) | 0.617 | 0.661 | 0.660 | 0.462 | 0.466 |
| 31 (Unspun) | 0.000 | 0.614 | 0.585 | 0.459 | 0.441 |
| % Interfer- | 100% | 7% | 11% | 0% | 5% |
| ence | | | | | |
| *The term “unspun” refers to an improperly prepared serum or plasma sample which was tested directly in the free PSA assay described above. The term “spun” refers to the same sample, which was re-centrifuged prior to testing. | | | | | |
| **% Interference = (Free PSA concentration from Spun sample-Free PSA concntration from Unspun Sample)/(Free PSA concentration from Spun sample) × 100 Although the addition of either polyhistidine (M.W. 13,200 daltons) or polyarginine | | | | | |
| # (M.W. 8,500 daltons) at a concentration of 0.025% also reduced interference, these concentrations interfered with the assay sensitivity. Lower concentrations of these poly-amino acids, however, may be effective at eliminating interference without affecting assay sensitivity. | | | | | |

### Example 4

### Effect of Polycations in the ARCHITECT total PSA Assay

The general procedure of the ARCHITECT total PSA assay is essentially as described for the free PSA assay in Example 3, with the exception that the paramagnetic microparticles are coated with a monoclonal antibody that binds to both free and complexed PSA. Experiments were performed in which unspun samples were subjected to a total PSA assay that utilized a microparticle diluent containing dextran sulfate (at a concentration of 0.05%) or a poly-L-lysine of average molecular weight 5200 or 11,200 (in place of dextran sulfate) at a concentration of 0.005%. The results, shown in Table 3, demonstrate that poly-L-lysines of different average molecular weights are effective at decreasing interferences in total PSA measurements in unspun samples without interfering with or altering the high functional sensitivity of the total PSA assay.

TABLE 3
Unspun Values for Total PSA (ng/mL)

| | | | poly-L- | | poly-L- | | | |
|---|---|---|---|---|---|---|---|---|
| Sample | Dextran | | lysine | | lysine | | No poly- | |
| No. | Sulfate | % Int. | 5200 MW | % Int. | 11200 MW | % Int. | L-lysine | % Int. |
| 55 | 4.070 | −62 | 10.707 | −6 | 10.806 | −8 | 7.724 | −27 |
| 56 | 6.663 | −33 | 10.329 | −1 | 10.357 | −2 | 8.922 | −8 |
| 71 | 0.083 | −99 | 13.431 | −4 | 14.695 | 0 | 1.398 | −90 |
| 72 | 0.483 | −97 | 14.797 | −8 | 15.025 | −10 | 5.996 | −61 |
| 73 | 0.057 | −99 | 9.983 | −8 | 10.353 | −8 | 1.149 | −89 |
| 74 | 0.099 | −99 | 12.483 | −1 | 13.414 | 4 | 2.573 | −79 |
| 75 | 0.389 | −97 | 14.658 | −7 | 15.211 | −6 | 8.892 | −40 |
| 76 | 5.681 | −52 | 12.158 | −7 | 12.158 | −5 | 9.665 | −17 |
| Avg. | | −80 | | −5 | | −4 | | −51 |
| Int. | | | | | | | | |

1-26. (canceled)

27. A method for decreasing interferences which result in inaccurate readings in serum or plasma containing assay samples of specific binding assays comprising adding an effective amount of a large, unconjugated polycation to serum or plasma containing assay samples during the specific binding assay, wherein the large polycation is a polylysine, polyornithine, polybrene, or dimethyldiallylammonium chloride having a molecular weight of 3,000 daltons or greater.

28. The method of claim 1 wherein the large polycation is a polylysine, polyornithine, or polybrene.

29. The method of claim 28 wherein the large polycation comprises a polylysine with a molecular weight ranging between 5,200 and 11,200 daltons.

30. The method of claim 27 wherein the large polycation comprises polylysine with a molecular weight of 8,800 daltons.

31. The method of claim 27 wherein the specific binding assay measures thyroid stimulating hormone, free prostate specific antigen, alpha fetal protein, Hepatitis B core antibody, Hepatitis B surface antibody or human immunodeficiency virus.

32. The method of claim 27 wherein said specific binding assay is performed on a solid phase.

33. The method of claim 32 wherein said solid phase comprises paramagnetic microparticles.

34. A method for decreasing interferences which result in inaccurate readings in serum or plasma containing assay samples of a thyroid stimulating hormone specific binding assay comprising adding a large, unconjugated polycation to serum or plasma containing assay samples during the thyroid stimulating hormone specific binding assay, wherein the large polycation is a polylysine, polyornithine, polybrene, or dimethyldiallylammonium chloride having a molecular weight of 3,000 daltons or greater.

35. The method of claim 34 wherein the large polycation is a polylysine, or polybrene.

36. The method of claim 34 wherein the large polycation comprises a polylysine with a molecular weight ranging between 5,200 and 11,200 daltons.

37. The method of claim 36 wherein the large polycation comprises polylysine with a molecular weight of 8,800 daltons.

38. A method for decreasing interferences which result in inaccurate readings in serum or plasma containing assay samples of a thyroid stimulating hormone specific binding assay comprising:
a) forming a first complex by incubating a serum or plasma sample with paramagnetic microparticles coated with anti-β TSH antibody and an assay diluent which comprises a large, unconjugated polycation, for a time and under conditions which allow the thyroid stimulating hormone present in the sample to bind to the anti-β TSH antibody coated microparticles;
(b) forming a second complex by incubating the first complex with an acridinium labeled conjugate comprising an anti-α TSH antibody, for a time and under conditions which allow the conjugate to bind to the first complex;
(c) creating a chemiluminescent reaction in the second complex; and
(d) measuring the chemiluminescent reaction as relative light units wherein the amount of thyroid stimulating hormone in the plasma or serum sample is directly related to the measured relative light units,
wherein the large polycation is a polylysine, polyornithine, polybrene, or dimethyldiallylammonium chloride having a molecular weight of 3,000 daltons or greater.

39. A method for decreasing interferences which result in inaccurate readings in serum or plasma containing assay samples of a free prostate specific antigen specific binding assay comprising adding a large, unconjugated polycation to serum or plasma containing assay samples during the free prostate specific antigen specific binding assay, wherein the large polycation is a polylysine, polyornithine, polybrene, or dimethyldiallylammonium chloride having a molecular weight of 3,000 daltons or greater.

40. The method of claim 39 wherein the large polycation is a polylysine or polyornithine.

41. A method for decreasing interferences which result in inaccurate readings in serum or plasma containing assay samples of a free prostate specific antigen specific binding assay comprising:
(a) forming a first complex by incubating a serum or plasma sample with paramagnetic microparticles coated with an antibody specific for free PSA and an assay diluent which comprises a large, unconjugated polycation, for a time and under conditions which allow the free PSA present in the sample to bind to the antibody coated microparticles;
(b) forming a second complex by incubating the first complex with an acridinium labeled conjugate comprising an anti-PSA antibody, for a time and under conditions which allow the conjugate to bind to the first complex;
(c) creating a chemiluminescent reaction in the second complex; and
(d) measuring the chemiluminescent reaction as relative light units wherein the amount of prostate specific antigen in the plasma or serum sample is directly related to the measured relative light units,
wherein the large polycation is a polylysine, polyornithine, polybrene, or dimethyldiallylammonium chloride having a molecular weight of 3,000 daltons or greater.

42. An improved specific binding assay kit for plasma and serum samples comprising a solution containing a large, unconjugated polycation, wherein the large polycation is a polylysine, polyornithine, polybrene, or dimethyldiallylammonium chloride having a molecular weight of 3,000 daltons or greater.

43. The improved specific binding assay kit of claim 39 wherein the large polycation is a polylysine, or polybrene.

44. The improved specific binding assay kit of claim 42 wherein the specific binding assay measures thyroid stimulating hormone, free prostate specific antigen, alpha fetal protein, Hepatitis B core antibody, Hepatitis B surface antibody or human immunodeficiency virus.

45. An improved kit for detection of thyroid stimulating hormone comprising:
(a) mouse, monoclonal anti-β TSH coated microparticles;
(b) mouse, monoclonal anti-α TSH acridinium-labeled conjugate; and
(c) a modified TSH assay diluent comprising a large, unconjugated polycation,
wherein the large polycation is a polylysine, polyornithine, polybrene, or dimethyldiallylammonium chloride having a molecular weight of 3,000 daltons or greater.

46. An improved kit for detection of free prostate specific antigen comprising:
(a) mouse monoclonal anti-Free PSA coated microparticles in a diluent comprising a large, unconjugated polycation; and
(b) mouse monoclonal anti-PSA acridinium-labeled conjugate,
wherein the large polycation is a polylysine, polyornithine, polybrene, or dimethyldiallylammonium chloride having a molecular weight of 3,000 daltons or greater.

47. The kit of claim 46 wherein the large polycation is a polylysine or polyornithine.

48. A method for decreasing interferences which result in inaccurate readings in serum or plasma containing assay samples of a total prostate specific antigen specific binding assay comprising:
(a) forming a first complex by incubating a serum or plasma sample with paramagnetic microparticles coated with an antibody which binds both free and complexed PSA and an assay diluent which comprises a large, unconjugated polycation, for a time and under conditions which allow the PSA present in the sample to bind to the antibody coated microparticles;
(b) forming a second complex by incubating the first complex with an acridinium labeled conjugate comprising an anti-PSA antibody, for a time and under conditions which allow the conjugate to bind to the first complex;
(c) creating a chemiluminescent reaction in the second complex; and
(d) measuring the chemiluminescent reaction as relative light units wherein the amount of prostate specific antigen in the plasma or serum sample is directly related to the measured relative light units,
wherein the large polycation is a polylysine, polyornithine, polybrene, or dimethyldiallylammonium chloride having a molecular weight of 3,000 daltons or greater.
